(19) United States
(12) Patent Application Publication
(10) Pub. No.: US 2011/0065769 A1
(43) Pub. Date: Mar. 17, 2011

(54) Transdermal delivery of ionizable drugs

(76) Inventor: Ooi Wong, Fremont, CA (US)

(21) Appl. No.: 12/799,918

(22) Filed: May 3, 2010

Related U.S. Application Data
(60) Provisional application No. 61/215,166, filed on May 1, 2009.

(51) Int. Cl. A61K 31/40; A61K 31/34; A61P 9/00

(52) U.S. Cl. 514/423; 514/470

(57) ABSTRACT

Provided is a composition for transdermal administration. The composition comprises a therapeutic agent having a net charge at skin pH and a topical penetration enhancer having a net charge at skin pH opposite the net charge of the therapeutic agent. In another aspect, a method is provided for increasing the transdermal absorption of a therapeutic agent. The method comprises forming a therapeutic composition at skin pH by combining a therapeutic agent having a net charge at skin pH with a topical penetration enhancer having a net charge at skin pH opposite the net charge of the therapeutic agent, the ratio of topical penetration enhancer to therapeutic agent being sufficient to increase absorption of therapeutic agent compared to absorption of therapeutic agent in the absence of topical penetration enhancer, and administering the therapeutic composition to a patient in need thereof.

### RELATED APPLICATIONS

This application claims benefit of priority to Provisional U.S. Patent Application No. 61/215,166, entitled “Transdermal Delivery of Ionizable Drugs”, filed May 1, 2009; the aforementioned priority application being hereby incorporated by reference for all purposes.

### FIELD OF THE INVENTION

The invention relates to compositions and methods for the transdermal delivery of ionizable drugs.

### BACKGROUND OF THE INVENTION

The transdermal route of drug administration offers a number of advantages over more conventional routes of drug administration. For instance, a drug may be delivered to targeted tissues from adjacent skin areas. The transdermal route of drug administration also allows for a gradual, controlled release of drug into the systemic circulation. Since many drugs are poorly absorbed or delivered through the traditional routes of administration, the transdermal route provides an effective method of achieving improved bioavailability for those drugs. The transdermal route of drug administration is also advantageous since the administration of dermally administered drugs may be easily stopped should an undesirable side effect occur during therapy.

In spite of the foregoing advantages, there are limitations to the use of transdermal formulations. They cannot generally be used successfully with most polar drugs since those drugs tend to penetrate the skin too slowly. This characteristic is particularly noteworthy since most drugs are of a polar character. Therefore, improved compositions and methods for the transdermal delivery of polar drugs are needed.

### SUMMARY OF THE INVENTION

In one embodiment, a composition is provided for transdermal administration. The composition comprises a therapeutic agent having a net charge at skin pH and a topical penetration enhancer having a net charge at skin pH opposite the net charge of the therapeutic agent.

Another embodiment provides a method of increasing the transdermal absorption of a therapeutic agent. The method comprises forming a therapeutic composition at skin pH by combining a therapeutic agent having a net charge at skin pH with a topical penetration enhancer having a net charge at physiological pH opposite the net charge of the therapeutic agent. The ratio of topical penetration enhancer to therapeutic agent is sufficient to increase absorption of the therapeutic agent compared to absorption of therapeutic agent in the absence of topical penetration enhancer. The therapeutic composition is administered to a patient in need thereof

Other methods, features and advantages of the present invention will be or become apparent to one with skill in the art upon examination of the following detailed descriptions. It is intended that all such additional methods, features and advantages be included within this description, be within the scope of the present invention, and be protected by the accompanying claims.

### DESCRIPTION OF PREFERRED EMBODIMENTS

Before the present compositions and methods are described, it is to be understood that the invention is not limited to the particular methodologies, protocols, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The present invention relates to the use of ion pairing between a biodegradable skin permeation promoter (BSPP) and an ionizable drug to promote transdermal delivery of the drug. In one aspect, the present invention is directed to a composition for transdermal administration comprising a therapeutic agent having a net charge at skin pH and a topical penetration enhancer having a net charge at skin pH opposite the net charge of the therapeutic agent. In one embodiment the skin pH is 5.5. The topical penetration enhancer increases the transdermal absorption of the therapeutic agent by at least 5 times relative to absorption of the therapeutic agent in the absence of the topical penetration enhancer, or preferably by at least 10 times relative to absorption of the therapeutic agent in the absence of the topical penetration enhancer, or more preferably by at least 20 times relative to absorption of the therapeutic agent in the absence of the topical penetration enhancer, or more preferably by at least 50 times relative to absorption of the therapeutic agent in the absence of the topical penetration enhancer, or more preferably by at least 100 times relative to absorption of the therapeutic agent in the absence of the topical penetration enhancer.

In another aspect, the composition comprises a therapeutic agent which has a net positive charge and the topical penetration enhancer has a net negative charge. For example, the therapeutic agent may comprise a moiety —NR¹R², wherein R¹ and R² are independently selected from hydrogen and a C₁-C₆ alkyl, such as methyl or ethyl. In one embodiment, the topical penetration enhancer may have the structure AM-HM¹-HM²-LM, where AM is an anionic moiety, HM¹ is a hydrophilic moiety, HM² is a second hydrophilic moiety, and LM is a lipophilic moiety. Preferably the bonds between HM¹-HM² and between HM²-LM can be degraded by an esterase. In one embodiment, the lipophilic moiety, LM may be a saturated or unsaturated aliphatic acid, or an aliphatic acid with a mixture of saturated and unsaturated bonds. In one embodiment, HM² may comprise propylene glycol. In one embodiment, AM-HM¹-has the general structure ⁻OOC—(C₁-C₆ alkyl)-CO—, wherein the C₁-C₆ alkyl may comprise saturated and/or unsaturated bonds. In another embodiment, the topical penetration enhancer comprises the structure CH₃—(CH₂)ₘ—(CH═CH)ₓ−(CH₂)ₙ—CR₁H—CO—O—C(CH₃)H—CR₂H—O—CO—C(CH₃)H—(CH₂)_{y}—COO⁻ wherein R₁ and R₂ are either hydrogen or a C₁-C₆ alkyl group, such as methyl or ethyl; m is a whole number from 0 to 12; n is a whole number from 0 to 12; x is a whole number from 0 to 3; and y is a whole number from 0 to 5.

In another aspect, the present invention is directed to the use of ion pairing between a basic drug and an anionic biodegradable skin permeation promoter to form an ion-pair which enhances transdermal delivery of the drug. For example, the basic drug can be an ionizable drug molecule which exists as a cationic species at skin pH, such as a drug molecule which contains an amine moiety. The anionic biodegradable skin permeation promoter used with the basic drug is a molecule with a net negative charge at skin pH. In one embodiment the skin pH is 5.5. For example, the anionic biodegradable skin permeation promoter can be a molecule with the general structure:

lipophiloic moiety-hydrophilic moiety A-hydrophilic moiety B

The hydrophilic moiety B contains at least one functional group which possesses a net negative charge at skin pH. In addition, when the biodegradable skin permeation promoter is exposed to the skin, it is capable of being degraded by enzymes in the skin, such as esterases, into the components which make up the BSPP, i.e., the lipophilic moiety, hydrophilic moiety A and hydrophilic moiety B. The components of the BSPP are nontoxic to the subject being treated.

In another aspect, the present invention is directed to a composition wherein the therapeutic agent has a net negative charge and the topical penetration enhancer has a net positive charge. In one embodiment, the therapeutic agent comprises a moiety —COO⁻. In another embodiment, the topical penetration enhancer has the structure CM-HM¹-HM²-LM, where CM is a cationic moiety, HM¹ is a hydrophilic moiety, HM² is a second hydrophilic moiety, and LM is a lipophilic moiety. Preferably, the bonds between HM¹-HM² and between HM²-LM can be degraded by an esterase. In one embodiment, the lipophilic moiety is a saturated or unsaturated aliphatic acid, or an aliphatic acid with a mixture of saturated and unsaturated bonds. In another embodiment, HM² comprises propylene glycol. In still another embodiment, CM-HM¹-has the general structure R₁R₂N—(C₁-C₆ alkyl)-CO—, wherein the C₁-C₆ alkyl comprises saturated and/or unsaturated bonds and R₁ and R₂ are independently selected from hydrogen and C₁-C₆ alkyl, such as methyl or ethyl. In yet another embodiment, the topical penetration enhancer comprises the structure CH₃—(CH₂)ₘ—(CH═CH)ₓ—(CH₂)ₙ—CR₁H—CO—O—C(CH₃)H—CR₂H—O—CO—C(CH₃)H—(CH₂)_{y}—NR₃R₄ wherein R₁ and R₂ are either hydrogen or a C₁-C₆ alkyl group, such as methyl or ethyl; R₃ and R₄ are either hydrogen or a C₁-C₆ alkyl group, such as methyl or ethyl; m is a whole number from 0 to 12; n is a whole number from 0 to 12; x is a whole number from 0 to 3; and y is a whole number from 0 to 5.

In still another embodiment, the present invention is directed to the use of ion pairing between an acidic drug and a cationic biodegradable skin permeation promoter to form an ion-pair which enhances transdermal delivery of the drug. For example, the acidic drug can be an ionizable organic acidic drug molecule which exists as an anionic species at skin pH, such as a drug molecule which contains a carboxylic acid moiety. The cationic biodegradable skin permeation promoter used with the acidic drug is a molecule with a net positive charge at skin pH. In one embodiment the skin pH is 5.5. For example, the cationic biodegradable skin permeation promoter can be a molecule with the general structure:

lipophiloic moiety-hydrophilic moiety A-hydrophilic moiety B

The hydrophilic moiety B contains at least one functional group which possesses a net positive charge at skin pH. In addition, when the cationic biodegradable skin permeation promoter is exposed to the skin, it is capable of being degraded by enzymes in the skin, such as esterases, into the components which make up the BSPP, i.e., the lipophilic moiety, hydrophilic moiety A and hydrophilic moiety B. The components of the BSPP are nontoxic to the subject being treated. Exemplary BSPPs for use with acidic drugs are described in U.S. Pat. No. 7,514,574, the contents of which are hereby incorporated by reference. The BSPPs described in U.S. Pat. No. 7,514,574 have the general structure:

CH₃—(CH₂)ₘ—(CH═CH)ₓ—(CH₂)ₙ—CH(R₁)—CO—O—CH(CH₃)—CH(R₂)—O—CO—CH(CH₃)—(CH₂)_{y}—N(CH₃)₂

where R₁ and R₂ are independently hydrogen or alkyl, such as methyl or ethyl; m is a whole number from 0 to 12; n is a whole number from 0 to 12; x is a whole number from 0 to 3; and y is a whole number from 0 to 5. One example of such a BSPP useful as an ion pairing agent with acidic drugs is N,N-dimethylamino isopropyl, propylene glycol laurate, having the structure:

CH₂—(CH₂)₉—CH₂—CO—O—CH(CH₃)—CH₂—O—CO—CH(CH₃)—CH₂—N(CH₃)₂

In preparing the compositions of the present invention, several factors are typically considered with respect to the therapeutic agent and topical penetration enhancer to be used. These factors include the ionizable center of the therapeutic agent and topical penetration enhancer; the size and nature of the cationic and anionic sites; the solvent in which the therapeutic agent and topical penetration are prepared; the pH of the environment; the pKa of the ionizable species; the concentration of the neutralized ion-pair formed by the therapeutic agent and the topical penetration enhancer; the lipophilicity of the neutralized ion-pair formed by the therapeutic agent and the topical penetration enhancer, and any other appropriate influencing factor.

In another aspect, the present invention is directed to a method of increasing the transdermal absorption of a therapeutic agent. The method comprises forming a therapeutic composition at skin pH by combining a therapeutic agent having a net charge at skin pH with a topical penetration enhancer having a net charge at physiological pH opposite the net charge of the therapeutic agent, the ratio of topical penetration enhancer to therapeutic agent being sufficient to increase absorption of therapeutic agent compared to absorption of therapeutic agent in the absence of topical penetration enhancer. The therapeutic composition is administered to a patient in need thereof. Any of the therapeutic agents and topical penetration enhancers described above may be used in the method.

The animals and cells treated according to the methods of the present invention preferably are mammals and mammalian cells. The methods can be used in any mammalian species, including human, monkey, cow, sheep, pig, goat, horse, mouse, rat, dog, cat, rabbit, guinea pig, hamster and horse. Humans are preferred.

The compounds of the present invention can be delivered directly or in pharmaceutical compositions along with suitable carriers or excipients, as is well known in the art. For example, a pharmaceutical composition of the invention may include a conventional additive, such as a stabilizer, buffer, salt, preservative, filler and the like, as known to those skilled in the art. Exemplary buffers include phosphates, carbonates, citrates and the like. Exemplary preservatives include EDTA, EGTA, BHA, BHT and the like.

An effective amount of such agents can readily be determined by routine experimentation, as can the most appropriate formulation. Various formulations are available in the art. See, e.g., Gennaro, A. R., ed. (1995) Remington's Pharmaceutical Sciences.

The pharmaceutical compositions of the present invention may be manufactured by any of the methods well-known in the art, such as by conventional mixing, dissolving, granulating, dragee-making, levigating, emulsifying, encapsulating, entrapping, or lyophilizing processes. As noted above, the compositions of the present invention can include one or more physiologically acceptable carriers such as excipients and auxiliaries that facilitate processing of active molecules into preparations for pharmaceutical use.

Proper formulation is dependent upon the route of administration chosen. For transmucosal administration, penetrants appropriate to the barrier to be permeated are used in the formulation. Such penetrants are described herein.

Compositions of the present invention are administered transdermally, such as through a skin patch, or topically. The transdermal or topical formulations of the present invention comprise one or multiple penetration enhancers.

For any composition used in the present methods of treatment, a therapeutically effective dose can be estimated initially using a variety of techniques well known in the art. For example, in a cell culture assay, a dose can be formulated in animal models to achieve a circulating concentration range that includes the IC₅₀ as determined in cell culture. Dosage ranges appropriate for human subjects can be determined, for example, using data obtained from cell culture assays and other animal studies.

A therapeutically effective dose of an agent refers to that amount of the agent that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation and dosage should be chosen, according to methods known in the art, in view of the specifics of a subject's condition.

The amount of agent or composition administered will, of course, be dependent on a variety of factors, including the sex, age, and weight of the subject being treated, the severity of the affliction, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein, and are specifically contemplated.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

### Example 1

A transdermal patch is formed which comprises about 2-5% actenolol (a beta blocker) and about 2% lisinopril (an ACE9 inhibitor), together with polyacrylic acid to make 100%.

### Example 2

Skin permeation experiments for several drug candidates were carried out using shaved rat skin. The dorsal skin from a rat was excised and mounted on a side by side diffusion cell assembly with a contact area of 0.95 cm² and cell volume of 2.5 ml. The temperature of the diffusion cell was regulated at 37° C. The excised rat skin was clamped between the donor cell and the receiver with the dorsal stratum corneum facing the donor cell.

The model drug molecules were suspended in physiological phosphate buffer solution, of pH 7.4. The sampling volume of 1.0 ml was removed at the time point and the sampling volume was replaced with a fresh volume of the buffer solution. Skin flux of the drugs was measured in the absence or presence of the enhancer N,N-dimethylamino isobutyric acid propylene glycol mono laureate (also known as dodecanoic acid 2-(3-dimethylamethyl-propionyloxy)-propylester, or PerRx™).

### Rat Skin Flux Enhancement

Drug Candidates

| | ISMN | ISDN | INDOM |
|---|---|---|---|
| (Treatment) | (angina) | (angina) | (gout and arthritis) |
| | μg/cm²-hr | | |
| control | 40 | 20 | 0.5 |
| (control + 3% PerRx ™) | 700 | 40 | 7.0 |
| Flux enhancement | 18X | 2X | 14X |
| (S.F. Control + | | | |
| 3% PerRx ™)/ | | | |
| (S.F. control) | | | |

ISMN = Isosorbide mono nitrate; ionizable hydrophilic drug candidate
ISDN = Isosorbide di nitrate; non ionizable hydrophilic drug candidate
INDOM = indomitable; ionization hydrophobic drug candidate
Experimental conditions
Skin membrane: hairless rat skin
Donor cell concentration: suspension to keep the donor cell under sink condition: pH 7.4.

### DISCUSSION

The results in the table show a very large skin flux enhancement by addition of 3% PerRx™ compared to the control formulations in which there was absence of 3% PerRx™ at pH 7.4. It is clear that the skin flux for ISMN, an ionizable hydrophilic acidic drug, and INDOM, an ionizable hydrophobic acidic drug, were enhanced most. The Skin Flux for the non ionized molecule was enhanced insignificantly. This confirms the facilitated mechanism of previous work (Fleekero, Wong and Rytting, “Facilitated transport of acidic and basic in solutions through skin by a new enhancer, Pharmaceutical Research, 1989, vol 6, 443-446). The enhancement effect of the enhancers depends upon the strength of the ion-pairing formation power. The experimental conditions affecting the enhanced skin flux are the pH, the loading concentration of the charged species and the saturation point in the curve of skin flux versus loading concentration.

| | enhancement of indomethacin flux |
|---|---|
| Unsaturated cyclic ureas series | 2X |
| Aminoacetic acid series | 3-4X |
| (ionizable enhancer) | |

Ion-pairing has been shown to exist in the amino acetic acid series by Rytting et al using C¹³ NMR spectroscopy. So in the present work, it is believed that at pH 7.4 addition of the acetic acid functional group into polar head position will affect tremendously the physical chemical properties of molecules. For example, addition of 3% PerRx™ to the control formulation does not affect the aqueous solubility of the ISMN, but the skin flux of ISMN is increased 18× by this addition. See the results table.

It will be appreciated that, although specific embodiments of the invention have been described herein for purposes of illustration, various modifications may be made without departing from the spirit and scope of the invention. All such modifications and variations are intended to be included herein within the scope of this disclosure and the present invention and protected by the following claims.

We claim:

1. A composition for transdermal administration comprising a therapeutic agent having a net charge at skin pH and a topical penetration enhancer having a net charge at skin pH opposite the net charge of the therapeutic agent.

2. The composition of claim 1 wherein the skin pH is 5.5.

3. The composition of claim 1 wherein the topical penetration enhancer increases the transdermal absorption of the therapeutic agent by at least 5 times relative to absorption of the therapeutic agent in the absence of the topical penetration enhancer.

4. The composition of claim 1 wherein the topical penetration enhancer increases the transdermal absorption of the therapeutic agent by at least 10 times relative to absorption of the therapeutic agent in the absence of the topical penetration enhancer.

5. The composition of claim 1 wherein the topical penetration enhancer increases the transdermal absorption of the therapeutic agent by at least 20 times relative to absorption of the therapeutic agent in the absence of the topical penetration enhancer.

6. The composition of claim 1 wherein the topical penetration enhancer increases the transdermal absorption of the therapeutic agent by at least 50 times relative to absorption of the therapeutic agent in the absence of the topical penetration enhancer.

7. The composition of claim 1 wherein the topical penetration enhancer increases the transdermal absorption of the therapeutic agent by at least 100 times relative to absorption of the therapeutic agent in the absence of the topical penetration enhancer.

8. The composition of claim 1 wherein the therapeutic agent has a net positive charge and the topical penetration enhancer has a net negative charge.

9. The composition of claim 8 wherein the therapeutic agent comprises a moiety —NR₁R₂, wherein R₁ and R₂ are independently selected from hydrogen and a C₁-C₆ alkyl.

10. The composition of claim 9 wherein R₁ and R₂ are independently selected from methyl and ethyl.

11. The composition of claim 8 wherein the topical penetration enhancer has the structure AM-HM¹-HM²-LM, where AM is an anionic moiety, HM¹ is a hydrophilic moiety, HM² is a second hydrophilic moiety, and LM is a lipophilic moiety.

12. The composition of claim 11 wherein the bonds between HM¹-HM² and between HM²-LM can be degraded by an esterase.

13. The composition of claim 11 wherein the lipophilic moiety is a saturated or unsaturated aliphatic acid, or an aliphatic acid with a mixture of saturated and unsaturated bonds.

14. The composition of claim 11 wherein HM² comprises propylene glycol.

15. The composition of claim 11 wherein AM-HM¹-has the general structure ⁻OOC—(C₁-C₆ alkyl)-CO—, wherein the C₁-C₆ alkyl comprises saturated and/or unsaturated bonds.

16. The composition of claim 11 wherein the topical penetration enhancer comprises the structure CH₃—(CH₂)ₘ—(CH═CH)ₓ—(CH₂)ₙ—CR₁H—CO—O—C(CH₃)H—CR₂H—O—CO—C(CH₃)H—(CH₂)_{y}—COO⁻ wherein R₁ and R₂ are either hydrogen or a C₁-C₆ alkyl group; m is a whole number from 0 to 12; n is a whole number from 0 to 12; x is a whole number from 0 to 3; and y is a whole number from 0 to 5.

17. The composition of claim 1 wherein the therapeutic agent has a net negative charge and the topical penetration enhancer has a net positive charge.

18. The composition of claim 17 wherein the therapeutic agent comprises a moiety —COO.

19. The composition of claim 17 wherein the topical penetration enhancer has the structure CM-HM¹-HM²-LM, where CM is a cationic moiety, HM¹ is a hydrophilic moiety, HM² is a second hydrophilic moiety, and LM is a lipophilic moiety.

20. The composition of claim 19 wherein the bonds between HM¹-HM² and between HM²-LM can be degraded by an esterase.

21. The composition of claim 19 wherein the lipophilic moiety is a saturated or unsaturated aliphatic acid, or an aliphatic acid with a mixture of saturated and unsaturated bonds.

22. The composition of claim 19 wherein HM² comprises propylene glycol.

23. The composition of claim 19 wherein CM-HM¹-has the general structure R₁R₂N—(C₁-C₆ alkyl)-CO—, wherein the C₁-C₆ alkyl comprises saturated and/or unsaturated bonds and R₁ and R₂ are independently selected from hydrogen and C₁-C₆ alkyl.

24. The composition of claim 19 wherein the topical penetration enhancer comprises the structure CH₃—(CH₂)ₘ—(CH═CH)ₓ—(CH₂)ₙ—CR₁H—CO—O—C(CH₃)H—CR₂H—O—CO—C(CH₃)H—(CH₂)_{y}—NR₃R₄ wherein R₁ and R₂ are either hydrogen or a C₁-C₆ alkyl group; R₃ and R₄ are either hydrogen or a C₁-C₆ alkyl group; m is a whole number from 0 to 12; n is a whole number from 0 to 12; x is a whole number from 0 to 3; and y is a whole number from 0 to 5.

25. A method of increasing the transdermal absorption of a therapeutic agent, the method comprising forming a therapeutic composition at skin pH by combining a therapeutic agent having a net charge at skin pH with a topical penetration enhancer having a net charge at physiological pH opposite the net charge of the therapeutic agent, the ratio of topical penetration enhancer to therapeutic agent being sufficient to increase absorption of therapeutic agent compared to absorption of therapeutic agent in the absence of topical penetration enhancer, and administering the therapeutic composition to a patient in need thereof.

26. The method of claim 25 wherein the skin pH is 5.5.

27. The method of claim 25 wherein the topical penetration enhancer increases the transdermal absorption of the therapeutic agent by at least 5 times relative to absorption of the therapeutic agent in the absence of the topical penetration enhancer.

28. The method of claim 25 wherein the topical penetration enhancer increases the transdermal absorption of the therapeutic agent by at least 10 times relative to absorption of the therapeutic agent in the absence of the topical penetration enhancer.

29. The method of claim 25 wherein the topical penetration enhancer increases the transdermal absorption of the therapeutic agent by at least 20 times relative to absorption of the therapeutic agent in the absence of the topical penetration enhancer.

30. The method of claim 25 wherein the topical penetration enhancer increases the transdermal absorption of the therapeutic agent by at least 50 times relative to absorption of the therapeutic agent in the absence of the topical penetration enhancer.

31. The method of claim 25 wherein the topical penetration enhancer increases the transdermal absorption of the therapeutic agent by at least 100 times relative to absorption of the therapeutic agent in the absence of the topical penetration enhancer.

32. The method of claim 25 wherein the therapeutic agent has a net positive charge and the topical penetration enhancer has a net negative charge.

33. The method of claim 32 wherein the therapeutic agent comprises a moiety —NR¹R², wherein R¹ and R² are independently selected from hydrogen and a C₁-C₆ alkyl.

34. The method of claim 33 wherein R¹ and R² are independently selected from methyl and ethyl.

35. The method of claim 32 wherein the topical penetration enhancer has the structure AM-HM¹-HM²-LM, where AM is an anionic moiety, HM¹ is a hydrophilic moiety, HM² is a second hydrophilic moiety, and LM is a lipophilic moiety.

36. The method of claim 35 wherein the bonds between HM¹-HM² and between HM²-LM can be degraded by an esterase.

37. The method of claim 35 wherein the lipophilic moiety is a saturated or unsaturated aliphatic acid, or an aliphatic acid with a mixture of saturated and unsaturated bonds.

38. The method of claim 35 wherein HM² comprises propylene glycol.

39. The method of claim 35 wherein AM-HM¹-has the general structure ⁻OOC—(C₁-C₆ alkyl)-CO—, wherein the C₁-C₆ alkyl comprises saturated and/or unsaturated bonds.

40. The method of claim 35 wherein the topical penetration enhancer comprises the structure CH₃—(CH₂)ₘ—(CH═CH)ₓ—(CH₂)ₙ—CR₁H—CO—O—C(CH₃)H—CR₂H—O—CO—C(CH₃)H—(CH₂)_{y}—COO⁻ wherein R₁ and R₂ are either hydrogen or a C₁-C₆ alkyl group; m is a whole number from 0 to 12; n is a whole number from 0 to 12; x is a whole number from 0 to 3; and y is a whole number from 0 to 5.

41. The method of claim 25 wherein the therapeutic agent has a net negative charge and the topical penetration enhancer has a net positive charge.

42. The method of claim 41 wherein the therapeutic agent comprises a moiety —COO⁻.

43. The method of claim 41 wherein the topical penetration enhancer has the structure CM-HM¹-HM²-LM, where CM is a cationic moiety, HM¹ is a hydrophilic moiety, HM² is a second hydrophilic moiety, and LM is a lipophilic moiety.

44. The method of claim 43 wherein the bonds between HM¹-HM² and between HM²-LM can be degraded by an esterase.

45. The method of claim 43 wherein the lipophilic moiety is a saturated or unsaturated aliphatic acid, or an aliphatic acid with a mixture of saturated and unsaturated bonds.

46. The method of claim 43 wherein HM² comprises propylene glycol.

47. The method of claim 43 wherein CM-HM¹-has the general structure R₁R₂N—(C₁-C₆ alkyl)-CO—, wherein the C₁-C₆ alkyl comprises saturated and/or unsaturated bonds and R₁ and R₂ are independently selected from hydrogen and C₁-C₆ alkyl.

48. The method of claim 43 wherein the topical penetration enhancer comprises the structure CH₃—(CH₂)ₘ—(CH═CH)ₓ—(CH₂)ₙ—CR₁H—CO—O—C(CH₃)H—CR₂H—O—CO—C(CH₃)H—(CH₂)_{y}—NR₃R₄ wherein R₁ and R₂ are either hydrogen or a C₁-C₆ alkyl group; R₃ and R₄ are either hydrogen or a C₁-C₆ alkyl group; m is a whole number from 0 to 12; n is a whole number from 0 to 12; x is a whole number from 0 to 3; and y is a whole number from 0 to 5.
